(19) United States
(12) Patent Application Publication
(10) Pub. No.: US 2024/0120047 A1
(43) Pub. Date: Apr. 11, 2024

(54) SYSTEM AND METHOD FOR REMOTE MEDICAL DATA SENSING AND ANALYSIS

(71) Applicant: Vector Remote Care, LLC, Bend, OR (US)

(72) Inventors: Luke Wilson Chambers, Bend, OR (US); Kevin Christopher Hoffman, Bend, OR (US); Ryan Charles Michel, Bend, OR (US)

(21) Appl. No.: 17/960,262

(22) Filed: Oct. 5, 2022

(51) Int. Cl. G16H 15/00; G16H 10/60; G16H 40/67

(52) U.S. Cl. CPC G16H 15/00; G16H 10/60; G16H 40/67

(57) ABSTRACT

A system receives medical data from multiple physiological monitor devices in various forms and formats. A data normalization process identifies each attribute type in the medical data reported by each device and assigns a unique attribute identifier. All parameters associated with an attribute are also identified and assigned a unique parameter identifier. The data normalization process uses this information to create a standardized data set for analysis. A rules processing engine applies alert and reporting rules to the standardized data set to generate a patient report and alerts for patient data that is beyond thresholds defined by the alert rules. The system provides default alert rules that may be overridden by clinic rule or manufacturer rules. Artificial intelligence (AI) principles may be applied using training set(s) of data to teach the rules processing engine to use the AI alert rules for data analysis.

### BACKGROUND OF THE INVENTION

### Field of the Invention

The present invention is directed to medical data analysis and, more particularly, to a system and method for remote medical data sensing and analysis.

### DESCRIPTION OF THE RELATED ART

Medical data is collected in a variety of different technologies. Traditional medical sensors are connected directly to the patient and to a conventional medical device. For example, an electrocardiograph (ECG) uses multiple electrodes connected to the patient at predetermined locations on the body. The electrodes are connected to an ECG machine that amplifies and displays the waveform. In some environments, such as a hospital or clinic, multiple ECG machines for different patients may be connected to a centralized monitoring station to display the ECG data and waveforms and thereby permit remote monitoring of multiple patients. It is also possible to program the ECG machines to set alarm limits that will alert a hospital employee to any alarm conditions, such as an irregular heartbeat.

Other conventional medical-grade devices can provide more remote monitoring. For example, a Holter monitor is a small wearable ECG device that continuously collects and stores ECG data for a period of time, such as 24-48 hours. Afterwards, a device can analyze the stored ECG data to detect any abnormalities. In another example, a Cardiovascular Implantable Electronic Device (CIED), such as a Pacemaker or Implanted Cardioverter Defibrillator (ICD), generates ECG data and also generates data related to pacemaker signals or defibrillation signals that may be generated by the device.

Modern communication technologies have led to a proliferation of medical monitoring devices that are integrated into everyday devices. For example, watches such as an Apple Watch™, are capable of monitoring health-related activities of the wearer to determine ECG, heart rate, blood oxygen level, and level of physical activity. The device is also capable of analyzing the data and generating alarms when an abnormal condition is detected. Other devices, such as Kardiamobile,™ provide electrode pads that work in conjunction with a software application program on a conventional smartphone to provide a portable ECG machine. The Kardiamobile device can generate and analyze an ECG waveform to detect abnormalities. The ECG waveform and analysis can be transmitted to a medical facility (e.g., a cardiac clinic) via a direct cellular connection or via the Internet using a cellular connection of the smartphone or using a WiFi, Bluetooth, Ethernet, or other conventional form of network connectivity.

The problem with the multiple forms of remote monitoring is that the cardiac clinic cannot manually process the volume of data produced by remote cardiac devices. Due to the sheer volume of incoming data, cardiac clinics also struggle to recognize when patients are no longer connected and have stopped transmitting.

Therefore, it can be appreciated that there is a need for a system that can receive incoming data from a variety of data sources, process the data to create uniform sets of data to which analysis rules can be applied and alarm conditions detected and reported. The present invention provides this and other advantages, as will be apparent from the following detailed description and accompanying figures.

### BRIEF DESCRIPTION OF THE SEVERAL VIEWS OF THE DRAWING(S)

FIGS. 1A and 1B together illustrate a block diagram of a system constructed in accordance with the present disclosure.

FIG. 2 is a flowchart illustrating the operation of the system of FIGS. 1A-1B to define normalized data from different input devices.

FIG. 3 illustrates a general data format of normalized data for example attributes.

FIG. 4 illustrates a data format of normalized data for specific example of attributes in the form of an atrial fibrillation episode and a device battery attribute.

FIG. 5 is a flowchart illustrating the operation of the system of FIGS. 1A-1B to normalize data received from different input devices.

FIG. 6 is a flowchart illustrating the operation of the system of FIGS. 1A-1B to implement a rules processing engine that processes the normalized data of FIG. 5.

### DETAILED DESCRIPTION OF THE INVENTION

As will be described in detail below, the present disclosure provides a mechanism that analyzes cardiac data generated by one or more input devices and processes the data from the input devices to thereby generate patient reports, including reports on episodes identified by the devices that occur while the patient is being monitored.

An “input device” is defined herein as an FDA regulated device that provides data (e.g., physiological data) about a patient. Examples of input devices include, but are not limited to, an implanted device (e.g., a CIED) that monitors (e.g., loop recorder) and/or also provides therapy/shocks (e.g., pacemaker, ICD); wearable devices (e.g., holter monitors, Zoll Life Vest, Apple Watch, Fitbit, and the like); and other measuring devices, (e.g., KardiaMobile 6L).

An input device is capable of secure transmission of data to network endpoint(s) that optionally perform additional processing and then securely stores the device data (e.g., cloud endpoints). Examples of secure transmission include standard TLS type encryption; however other known forms of encryption can be satisfactorily implemented. Transmission examples include wired, wireless, local area networks (LAN), wide area networks (WAN), and cellular networks. A device may transmit to an intermediate device (e.g., a bedside monitor connected to a LAN) before ultimately being stored on a network endpoint. A device's stored data can be securely retrieved by an authenticated user through a network interface (e.g., a RESTFul web API).

An “episode” is defined herein as an arrhythmia event in the heart. Non-limiting examples of episodes are bradycardia, tachycardia, atrial fibrillation, post-ventricular contractions, and the like. The episodes detected by the input devices are described in terms of attributes.

An “attribute,” as used herein, can be any class of information that the device detects about the patient being monitored as well as operational parameters of the device itself. In addition, an attribute can include known information about the patient, for example medication(s) or chronic conditions. Examples of an attribute include, but are not limited to, episodes (e.g., an arrhythmia event in the heart), pacing and sensing values (e.g., atrial pacing of 50%), physiological measurements (e.g., thoracic impedance), device health (e.g., battery longevity), lead connection status, patient medication(s) (e.g., anti-coagulants), chronic conditions (e.g., persistent atrial fibrillation (AF)), and the like.

The term “provider” refers to a medical service provider, such as a medical doctor, who reviews and signs the medical reports generated by the system described herein. A “technician” refers to an individual who is qualified or credentialed to preview/triage the reports prior to the delivery to the provider. The technician may find that the data is not actionable (i.e., no action is required in response to the data) or that the data is simply noise. The technician determines whether or not the data transmission from the input device is worthy of a care action or is a billable event (a billable event requires review and signature from a provider). A “user” refers generically to a person using the system described herein. A technician is a user that modifies the reports and prepares them for the provider. In turn, the provider is a user that reviews the prepared reports. In some settings, a “manager” is a user that oversees the workflow to make sure that the process is flowing smoothly.

A “clinic” refers to a medical facility, such as a hospital, specialty clinic, or the like. A “manufacturer” is an entity that manufacturers one or more input devices.

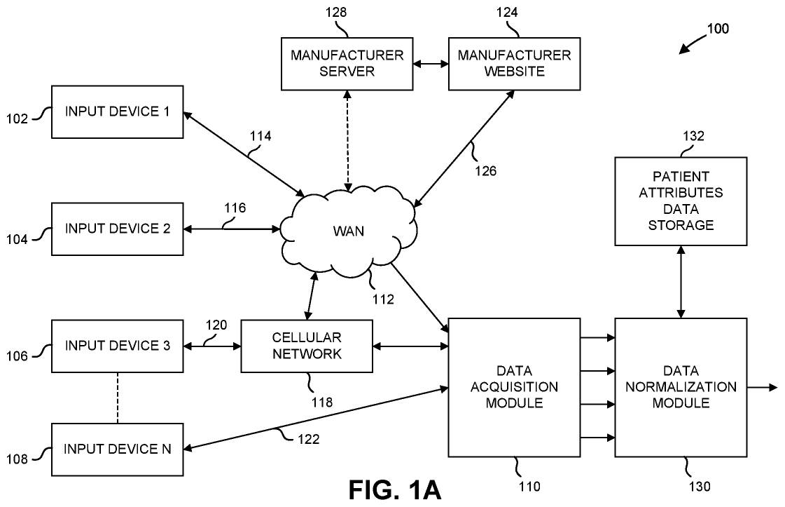

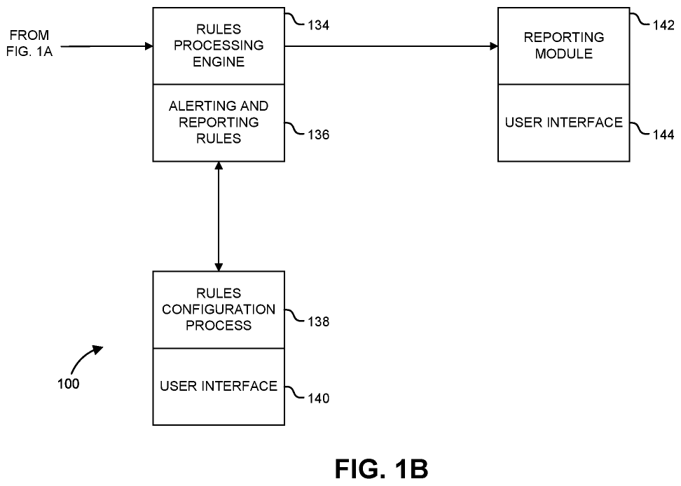

The present disclosure is embodied in a system 100 illustrated in the functional block diagram of FIGS. 1A-1B. In an exemplary embodiment, the system 100 is a cardiac monitor and data analysis system. In FIG. 1A, a plurality of devices 102-108 generically illustrate various forms of cardiac monitoring devices.

Those skilled in the art will appreciate that the devices 102-108 represent a broad range of medical monitoring devices that may be wearable (e.g., a wristwatch worn by the patient), attachable (e.g., electrodes attached to the patient), touchable (e.g., the patient places fingers on a sensor pad), implantable (e.g., an implantable pacemaker or ICD). For the sake of clarity, the specific connections between the patient and the devices 102-108 are not illustrated in FIG. 1A.

Data collected from the patient is routed to a manufacturer website 124 associated with each of the respective devices 102-108. Those skilled in the art will appreciate that the devices 102-108 themselves and the manufacturer website 124 are not part of the system 100, but communicate with the inventive system to provide patient data thereto. As will be described in greater detail below, the plurality of devices 102-108 are coupled to the manufacturer website 124 using a variety of conventional techniques. The data is supplied to the manufacturer website 124 using secure connections.

In turn, a data acquisition module 110 accesses the manufacturer website 124 to retrieve the patient data. Those skilled in the art will appreciate that the devices 102-108 each provide respective output data streams in their own format. In an exemplary embodiment, the data acquisition module 110 routes the incoming data streams to a data normalization module 130.

In addition to the data generated by the devices 102-108, the system includes a patient attributes data storage 132. Patient attributes include patient data, such as chronic conditions, medication history, and the like. The patient attributes are part of the patient medical records and are typically entered into the patient attributes data storage 132 by medical personnel (e.g., a technician) in the clinic or extracted from clinic patient records. The patient attributes are provided to the data normalization module and incorporated into the normalized data.

The data normalization module 130 transforms the attribute data (e.g., episode data, pacing data, device status data, patient data, and the like) from each of the different devices 102-108, and from the patient attributes data storage 132, into a uniform standard format to permit further data analysis on a standardized data set. Details of the data normalization module 130 are provided below. The uniform standard data set is analyzed by a rules processing engine 134, illustrated in FIG. 1B. The rules processing engine 134 applies a set of alerting and reporting rules 136 to the uniform standard data set. As will be discussed in detail below, in one aspect, the alerting and reporting rules 136 define alerting thresholds. For example, one threshold may define an alerting level for atrial fibrillation that can be based on defined parameters such as the percentage of heartbeats that are abnormal and the percentage of time the abnormal heartbeats occur during a defined time range (e.g., 12 hours or 24 hours). The percentage of heartbeats that are abnormal divided by the defined time range is referred to as the “burden.” In this example, the burden can also have a defined threshold value.

The rules processing engine 134 also generates text summaries of the data analysis after the application of the alerting rules. In a second aspect, the alerting and reporting rules 136 provide the rules processing engine 134 with text summaries that identify the detected episode and provides additional information. A text summary is a narrative that accompanies the report of each episode. For example, the text summary may be “Tachycardia detected” and include heartrate data. The text summary may also apply a selected background color to provide a visual alert to the user. For example, a heartrate may be shown with a green, yellow, or red background to indicate levels of urgency related to the alerting, text summary, and associated data.

As will be discussed in greater detail below, the alerting and reporting rules 136 are initially provided with default values. In this manner, the system 100 can operate without the need for customized configuration. However, the system 100 also includes a rules configuration process 138 and a user interface 140 that permits a clinic to define their own alerting rules.

Similarly, the alerting and reporting rules 136 are initially provided with default text summaries. In this manner, the system 100 will operate without the need for customized configuration. However, the rules configuration process 138 and the user interface 140 permits a clinic to define its own structured text summaries. Following the data analysis and application of alerting rules and text summaries to the standardized data set, the system 100 provides a reporting module 142 that serves as a repository of reports. The reporting module 142 does not generate reports, but permits the clinic user, such as the technician, to triage, search, view, and edit the reports before sending the reports to a provider for their review and signature. The user interface 144 permits user access to the reporting module 142 to perform these tasks.

Returning again to FIG. 1A, the data acquisition module 110 provides indirect access to the data from the devices 102-108. The current customary technique for communication with the devices 102-108 is via the manufacturer website 124, which is coupled to a wide area network (WAN) 112, such as the Internet, via a communication link 126. Data from the devices 102-108 is provided to the manufacturer website 124. The manufacturer also has a manufacturer server 128 on which data received from the devices 102-108 is stored. The manufacturer server 128 queues the data for retrieval by the system 100. It should be noted that FIG. 1A only illustrates a single manufacturer website 124 and a single manufacturer server 128. While a single manufacturer website 124 and a single manufacturer server 128 is sufficient for multiple devices (e.g., devices 102-104) made by that manufacturer, typically, each device manufacturer will have its own website and server for the respective devices made by that manufacturer. For the sake of simplicity, FIG. 1A has omitted multiple manufacturer websites and multiple manufacturer servers.

The data collection begins with the input devices 102-108. As noted above, data transmission from the input devices 102-108 can include wired transmission, wireless transmission, local area networks (LAN), wide area networks (WAN), and cellular networks. Furthermore, the devices 102-108 may transmit to an intermediate device before transmission to the manufacturer website 124.

For example, the device 102 is coupled to the manufacturer website 124 via the WAN 112. As illustrated in FIG. 1A, the device 102 is coupled to the WAN 112 using a wireless communication link 114. The wireless communication link 114 may be implemented by a number of different technologies, such as Bluetooth, Wi-Fi, or the like. For the sake of clarity, intermediate components, such as a wireless access point (WAP) are omitted. In contrast, the device 104 may be coupled to the WAN 112 via a hardwire communication link 116 such as an Ethernet connection, a VoIP connection, or the like. Again, intermediate components, such as a network access point (NAP) or an intermediate LAN connection to a NAP, are omitted from FIG. 1A for the sake of clarity.

In another example, the device 106 may be coupled to the manufacturer server 128 via a cellular network 118. The device 106 is coupled to the cellular network 118 via a cellular communication link 120. For the sake of clarity, intermediate components, such as a cell tower and cellular network infrastructure are omitted from FIG. 1A. In turn, the cellular network 118 may be coupled to the manufacturer server 128 via telephone network communication links or via the WAN 112, as illustrated in FIG. 1A.

In yet another example, the device 108 may be directly coupled to the data acquisition module 110 using a cable 122 or other conventional component. This implementation is appropriate if the system 100 is implemented in a clinical setting, such as a hospital. Data may also be delivered to the data acquisition module 110 via other forms of data storage, such as CDs, DVDs, magnetic storage devices, memory cards (e.g., MicroSD card), and the like. The present disclosure is not limited by the type of input device or the particular communication link used to couple the devices 102-108 to the manufacturer website 124 or the data acquisition module 110.

In operation, each provider has security credentials that permit access to each manufacturer website 124 and manufacturer server 128 to retrieve data. The system 100 uses these credentials to poll the manufacturer website 124 and access the manufacturer server 128 to retrieve data for a particular patient. The queued data in the manufacturer server 128 is provided to the data acquisition module 110 via a secure connection. Polling intervals may be determined by each clinic or individually by each provider. Thus, the data acquisition module 110 provides both authentication with the manufacturer website 124 and data transfer from the manufacturer server 128 to the system 100.

In the embodiment described above, the system 100 operates with polling to pull the data from the manufacturer server 128 at a rate determined by a user of the system. However, the system 100 can also be configured to operate with push technology so that a data transfer can be initiated by the manufacturer. For example, the system 100 can implement a Message Queuing Telemetry Transport (MQTT) protocol. This is sometimes referred to a publish-subscribe protocol where the manufacturer website 124 publishes a data availability notice that is received by all subscribers. In this embodiment, the data acquisition module 110 functions as a subscriber and monitors an available data port to detect the publish notice. Upon receipt of the publish notice, the data file is transferred from the manufacturer website 124 or manufacturer server 128 to the data acquisition module 110 via the WAN 112. The advantage of this approach is that the manufacturer can initiate a data transfer periodically or upon detection of certain conditions, such as detection of an episode. Those skilled in the art will appreciate that other communication protocols can be satisfactorily employed by the system 100.

It should be noted that the devices 102-108 each provide respective data streams in their own format. This may include variations in the data itself, any text summary, accompanying the data, alerting thresholds, and data form (e.g., PDF, text file, and the like). In an exemplary embodiment, the data acquisition module 110 routes the incoming data streams, each in its own format, to the data normalization module 130.

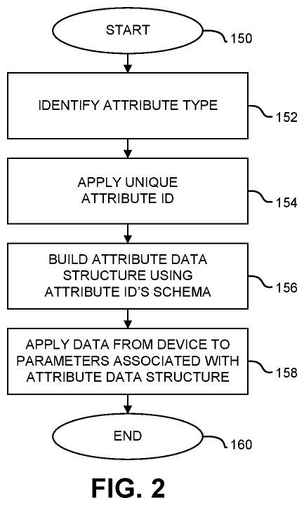

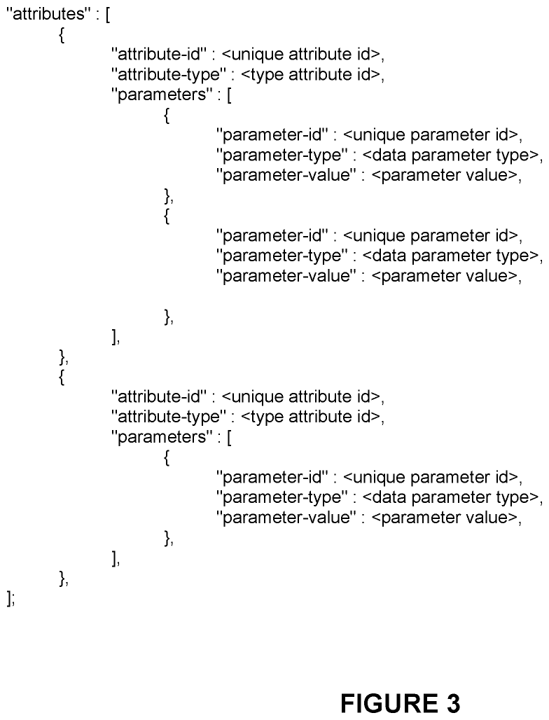

Prior to operation with real-time clinical data, the system 100 undergoes a setup operation in which all data generated by each of the devices 102-108 undergoes a normalization analysis. An exemplary embodiment of the normalization analysis process is illustrated in the flowchart of FIG. 2 where, at a start 150 a data set from one of the devices 102-108 is provided to the data normalization module 130 (see FIG. 1A). In step 152, a type of attribute is identified, and in step 154 a unique attribute ID code is assigned to the attribute type. For example, an episode type, such as the abnormal heart rhythm atrial fibrillation (AT/AF), is assigned a unique attribute ID code, such as a multi-digit integer value. Because the attribute ID code is used internally by the system 100 to identify a particular attribute type, the attribute ID code does not need to be in human readable form. Each attribute type has associated zero or more parameters that are related to the identified attribute type and provided by the particular device that detects the attribute. FIG. 3 provides a generic example of the format used by the system 100 to identify an attribute and define parameter values associated with the particular attribute. Each parameter associated with a particular attribute is also assigned a unique parameter ID code. As with the attribute ID code, the parameter ID code does not need to be in human readable form.

In the example of FIG. 3, two different attributes are defined. Each attribute is then associated with a unique ID value (e.g., “<unique attribute id>”). Each attribute also has zero or more parameters that are measured by a particular one of the devices 102-108. Each parameter for a particular attribute is assigned a unique parameter ID value (e.g., “<unique parameter id>”). In the example of FIG. 3, the first attribute has two associated parameters, and the second attribute has only one associated parameter. Each parameter definition also includes format data associated with the parameter. The data type of the parameter is assigned in the parameter type field (e.g., “<data parameter type>”) and the parameter's data value is assigned in the parameter value field (e.g., “<parameter value>”).

Those skilled in the art will appreciate that different ones of the devices 102-108 detect different types of episodes and that the attributes for each of the devices may generate a different number of parameters and different types of parameters. For example, an external wearable device, such as a watch, can detect different types of episodes and may therefore generate a different number of parameters and different parameter types for a given episode than an implantable monitor/pacemaker. Thus, the episode definitions and parameter descriptions will vary from device to device and from one episode type to another episode type.

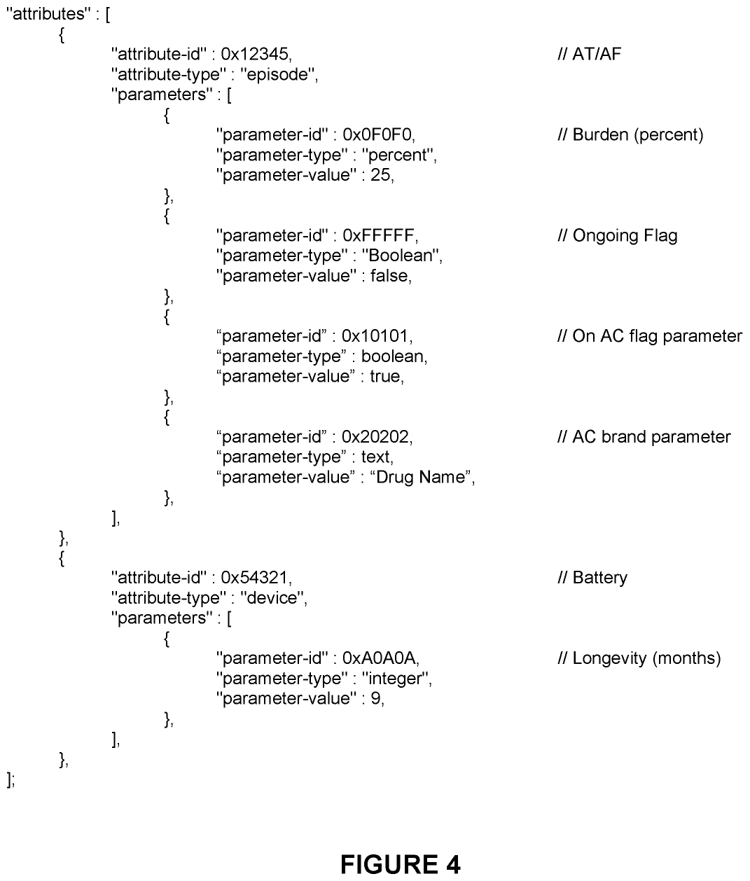

FIG. 4 provides a specific attribute example of the format used by the system 100 to identify an attribute for an atrial fibrillation (AT/AF) episode type (e.g., “attribute-type”: “episode”) and to define parameter values, such as the burden, associated with this particular episode type. FIG. 4 also illustrates additional parameter values related to patient data provided by the patient attribute data storage 132 (see FIG. 1A). In the present example, the patient medication status is associated with AT/AF. FIG. 4 illustrates a parameter for anti-coagulant (AC) and a flag that is set if the patient is on AC medication. A related parameter identifies the specific type of AC medication.

FIG. 4 also illustrates additional parameter values that indicate if the episode is ongoing at the time of the observation from each of the devices 102-108. Although FIG. 4 only illustrates six associated parameters, other parameters, such as the duration of the longest atrial episode, may be associated with the AT/AF episode type. For example, a first of the devices 102-108 that can generate four parameters associated with atrial fibrillation episode type will include four different parameter values, one for each of the four unique parameter IDs defined for the attribute. A second of the devices 102-108 that can generate only two parameters associated with atrial fibrillation will generate two different parameter values out of the four unique parameter IDs defined for the attribute, leaving the other two parameters undefined. In addition, patient data parameters, such as medication status, is provided by provided by the patient attribute data storage 132 (see FIG. 1A).

It should be noted that the same <unique attribute id> is assigned to the same attribute no matter which of the devices 102-108 generates the attribute. Similarly, the same <unique parameter id> is assigned to the same parameter no matter which of the devices 102-108 generates the parameter. For example, an atrial fibrillation (AT/AF) episode type (e.g., “attribute-type”: “episode”) and parameter values, such as the burden, will be assigned the same <unique attribute id>ID code and the same <unique parameter id>ID code no matter which of the devices 102-108 generates the data. With this approach, all normalized data will have the same attribute ID code for a particular attribute and the same parameter IDs for associated parameters no matter which of the devices 102-108 generated the data. This simplifies the application of rules to the data.

In summary, the system 100 takes data from any device and produces an array of N “attributes” that are derived from the data in the transmission from all devices. For any particular one of the devices 102-108, the data normalization module 130 receives the data and generates an output that is a subset of the N attributes. Attributes include episode data, as described above, but also include other classes of information including, but not limited to, device information, notifications, therapy/shocks, pacing, patient information, and the like.

For example, device information may include battery status, expected battery life (i.e., longevity) or information about other operational parameters of the device, such as pacing signals applied to the heart by an implantable pacemaker. Thus, battery status may be classified as an attribute, and longevity is a parameter. The medical device manufacturer or individual clinics may set an alert threshold for battery replacement. For example, one clinic may set a 1-year expected life remaining for the battery as a threshold alert to schedule a procedure to implant a new battery, while a different clinic may set the threshold alert at six months. In an example of an attribute with zero parameters, an alert condition is detected by one of the devices 102-108 when the lead impedance is above an impedance threshold (e.g., indicating a loose or disconnected lead). The attribute ID simply indicates a “Lead Impedance Alert” that provides an alert to the technician or provider with no associated parameter.

Similarly patient information can also be normalized as attributes to which alert rules may be applied. For example, a patient with known AT/AF is often on AC drugs to prevent the formation of blood clots. If the system 100 detects an episode of AT/AF, but has no information regarding patient AC drugs, there may be a higher alert level (e.g., a yellow alert or a red alert) depending on the severity of the AT/AF. However, if the patient is known to be on AC drugs, the AT/AF episode might not even be an alertable event because there is little or no risk to the patient. Thus, the patient information, provided by the patient attribute data storage 132 (see FIG. 1A), can be used by the system 100 to alter the episode analysis.

Returning to FIG. 2, in step 156, the data normalization module 130 defines an attribute data structure for all attributes and parameters associated with the particular episode type that are generated by the one of the devices 102-108 currently being analyzed. In step 158, the data normalization module 130 defines a data structure and format for each of the parameters associated with the particular attribute type and all the parameters associated with a particular attribute type. This is described above in the examples of FIGS. 3 and 4.

Steps 152-158 are repeated for each attribute type so that all attribute types capable of detection by the particular ones of the devices 102-108 are uniquely identified and all parameters associated with each attribute type are uniquely identified. This process is then repeated for all the devices 102-108 and the process ends at 160. As new medical devices appear in the marketplace or are added to the system 100, the process of FIG. 2 can be repeated.

In live data collection operation following the definition process, a real-time data stream is generated by each of the devices 102-108 and routed to the manufacturer server 128, as described above. In an exemplary embodiment, the system 100 logs onto the manufacturer website 124 (see FIG. 1A) and accesses the manufacturer server 128 to retrieve a data report from an individual patient. As noted above, the data retrieval is typically performed on a manufacturer-by-manufacturer basis. For example, devices 102-104 may be made by a first manufacturer. It is possible for the system 100 to retrieve the patient data from both devices by accessing the manufacturer server 128 for the particular manufacturer of devices 102-104. The system 100 must access a different manufacturer website 126 and manufacturer server 128 for a device (e.g., the device 106) made by that manufacturer and must further access a third manufacturer website 126 and manufacturer server 128 for a device (e.g., the device 106) made by the third manufacturer. This data retrieval process is repeated for all manufacturers until data by all the devices 102-108 has been retrieved.

With a different system architecture, the patient data from the devices of different manufacturers may be periodically forwarded to a single data server for retrieval by the data acquisition module 110 (see FIG. 1A). This approach can simplify the acquisition of data by the system 100 because the data acquisition module 110 can retrieve a patient's data from all the devices 102-108 by accessing a single server.

In a typical data retrieval operation, the provider (e.g., a medical doctor) is provided with authentication credentials, such as a user ID and password, by each manufacturer to permit access to the manufacturer server 128. The access may be direct or via the manufacturer website 124. The authentication credentials are provided to the system 100 for each manufacturer to permit automatic data retrieval by the system. Other known forms of user authentication, such as the MQTT protocol discussed above, may be satisfactorily implemented by the system 100.

The data retrieval process can be automatically initiated at predetermined time intervals, which may be altered from one patient to another or in response to the patient data itself. For example, detection of an episode with parameter values above a predetermined threshold may cause the system 100 to decrease the time intervals for the patient so that data is collected more frequently. Conversely, data below a threshold for a predetermined time may cause the system 100 to increase the time intervals so that data is collected less frequently.

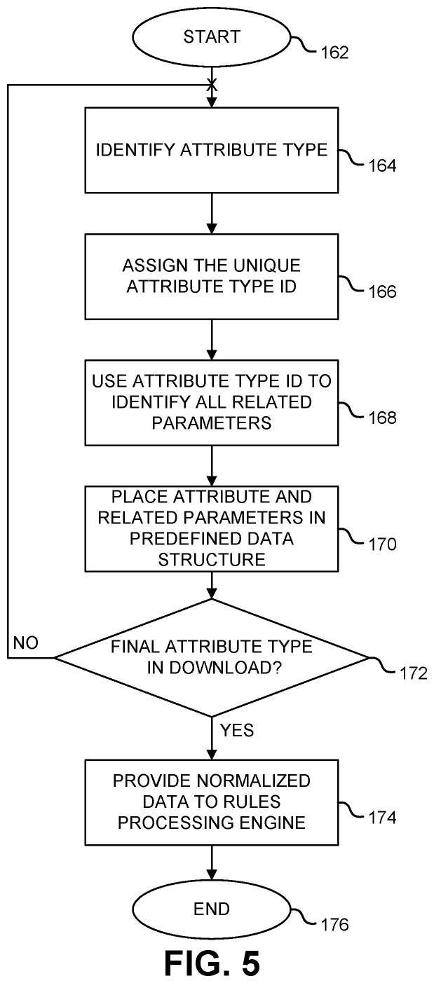

The flowchart of FIG. 5 illustrates an example of the data normalization process performed by the data normalization module 130 for actual patient monitoring data received from the devices 102-108 (see FIG. 1A). At a start 162, the data from the devices 102-108 is acquired by the data acquisition module 110 in the manner described above and provided to the data normalization module 130. In step 164, the data normalization module 130 identifies an attribute type. In step 166, the data normalization module 130 assigns the unique attribute type ID previously defined for that attribute type and, in step 168, uses the unique attribute type ID to identify all related parameters. As discussed above, the devices 102-108 may generate a different number of parameters and/or different parameter types for a given attribute type. Thus, a particular one of the devices 102-108 may generate from zero to all of the parameters associated with the identified attribute type. Any associated parameters not generated by the particular on the devices 102-108 are left undefined.

In step 170, the data normalization module 130 places the identified attribute and all related parameters in the predefined data structure and format for the identified attribute type, such as that illustrated in FIGS. 3 and 4.

In decision 172, the data normalization module 130 determines if the final attribute type has been received from the data acquisition module 110. If not, the result of the decision 172 is NO, and the process returns to step 164 to identify another attribute type in the data. If the final attribute type has been processed in step 170, the result of the decision 172 is YES. If so, the data normalization module 130 provides the normalized data to the rules processing engine 134 (see FIG. 1B) in step 174 and the process end at 176. Those skilled in the art will appreciate that the process of FIG. 5 is repeated for the data received from each of the devices 102-108. Those skilled in the art will appreciate that other data identification and formatting may be satisfactorily used by the system 100.

Thus, the data normalization module 130 has the ability to receive data generated by all the devices 102-108, in real-time and in all its various forms, and to create a set of data with a uniform standard format that identifies each attribute type and all parameters relevant to each attribute type. Each attribute type is given a name (text), a unique attribute ID, and zero or more associated parameters, each with its own unique parameter ID code. As discussed above, patient data from the patient attribute data storage 132 is also provided to the data normalization module 130 to provide normalized data that includes any relevant patient data.

The data in the uniform standard format generated by the data normalization module 130 is then analyzed by the rules processing engine 134, illustrated in FIG. 1B. The rules processing engine 134 uses the alerting and reporting rules 136 to drive decision making for structured reporting and alerting.

The alerting and reporting rules 136 define alert thresholds for the reporting of episodes. In one example, numeric data (e.g., heart rate) is provided in a report for an episode type detected by the devices 102-108 and shown on a computer display (not shown) or a report printed on a printer (not shown). The system 100 can define multiple levels of “urgency” in reporting episodes using multiple thresholds. This may include, for example a heartrate that is above a selected threshold rate (i.e., tachycardia) or below a selected threshold rate (i.e., bradycardia). The urgency may also be indicated by a color indicator where, for example, a green background color indicates a value within a normal range. A yellow background color indicates a value marginally beyond (e.g., above or below) the normal range and a red background color indicates a value significantly beyond (e.g., significantly above or below) the normal range. The color urgency indicators provide an easily identified abnormality for a reviewer.

In another example of alerting and reporting rules 136 operating with multiple thresholds, if the AT/AF burden level is 90%, the alerting rules may generate a red alert (i.e., a red background color) to indicate a significant abnormality, and a burden ≥1% and <90% can generate a yellow alert, and any other burden level (i.e., <1%) results in a green background indicating no alert. As noted above, the alerting rules also take other attributes, such as patient using AC drugs, into account when determining the alert condition.

The alerting and reporting rules 136 may include threshold values that are defined in a hierarchical fashion depending on the source of the threshold values. For example, the devices 102-108 typically have alerting threshold values defined by the particular manufacturers. However, the alerting and reporting rules 136 may include default alert threshold values that permit the system 100 to operate satisfactorily using only the default alert thresholds. In an exemplary embodiment, default rules (both structured reporting and alerting) may be created by the developer of the system 100 as a certified independent diagnostic and testing facility. In a hierarchical fashion, the default rules created by the developer of the system 100 override any threshold values provided by a manufacturer of the devices 102-108.

However, the rules configuration process 138 and the user interface 140 permit a clinic to define its own alert thresholds. In a hierarchical fashion, clinic-defined alert thresholds will override default alert thresholds. Within a clinic, the alerting rules are typically set by an expert individual or committee. The individual is typically a provider and could, for example, be a department head, such as an electrophysiologist. In one embodiment, the clinic can provide clinic-defined alert thresholds to the developer of the system 100 for integration into the system. Alternatively, the clinic may independently define alert thresholds using the user interface 140. In either case, clinic-defined alert thresholds will override default alert thresholds.

The structured reporting comprises summary text that will accompany the full data transmission from the devices 102-108. The alerting and reporting rules 136 also includes default summary texts that permit the system 100 to operate satisfactorily using only default summary texts. In the hierarchical fashion described above, the default summary texts created by the developer of the system 100 override any summary texts provided by a manufacturer of the devices 102-108.

However, the rules configuration process 138 and the user interface 140 permit a clinic to define its own summary texts. In the hierarchical fashion described above, clinic-defined summary texts will override the default summary texts created by the developer of the system 100. For example, a default summary text for AT/AF may be “AT/AF Detected, Burden 25%” (where the 25% burden value is provided as a parameter associated with the AT/AF episode attribute) while a particular clinic may wish to override the default summary text and display “Possible AT/AF Detected, Burden 25%.” In one embodiment, the clinic can provide clinic-defined summary texts to the manufacturer of the system 100 for integration into the system. Alternatively, the clinic may independently define summary texts using the user interface 140. In either case, clinic-defined summary texts will override default summary texts.

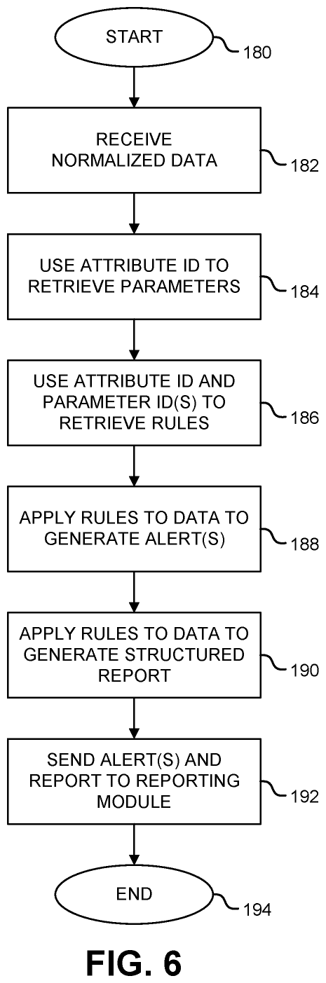

The operation of the rules processing engine 134 is illustrated in the exemplary flowchart of FIG. 6 where at a start 180, the data from the devices 102-108 has been normalized by the data normalization module 130 (see FIG. 1A). In step 182, the rules processing engine 134 receives the normalized data. As noted above, the normalized data may include data from different ones of the devices 102-108 that relate to the same attribute. For example, an episode of AT/AF may be detected by three different ones of the devices 102-108. Each device of the devices 102-108 capable of detecting an AT/AF episode will report the attributes and associated parameters related to the AT/AF episode. In one embodiment, the rules processing engine 134 processes the data independently from each device and generates separate reports for each device. This means that the same episode will be reported multiple times.

Alternatively, the rules processing engine 134 can analyze the data from one of the devices 102-108 that generates the greatest number of attributes related to the particular episode. In this manner, the rules will be applied against a larger amount of data reported by one of the devices 102-108. In yet another alternative, the system 100 can combine data from the multiple devices that have detected the same episode. In the above example, the AT/AF data from the three devices can be normalized and then combined for analysis by the rules processing engine 134. The report on the AT/AF episode is based on data from the three devices that detected the episode.

Returning to FIG. 6, in step 184, the rules processing engine 134 uses the unique attribute ID to retrieve the associated parameters. In step 186, the rules processing engine 134 uses the unique attribute IDs to retrieve the rules that are used to analyze the data associated with the identified attribute. If an attribute's rule relies on its parameters for alerting and/or structured reporting, the attribute's parameter IDs are used to retrieve the rules that are used to analyze the data for each of the parameters associated with the identified attribute. As previously discussed, the alerting and reporting rules 136 stores the rules data that can be applied to the normalized data. The rules processing engine 134 applies the rules to the normalized data to determine that some parameter is within a normal range or if the parameter is beyond (above or below) the normal range.

In step 188 the rules from the alerting and reporting rules 136 are applied to the data initially generated by the devices 102-108 to generate alerts based on the rules and the data. In step 190, the rules from the alerting and reporting rules 136 are applied to the data from the alert analysis performed in step 188 to generate a structured report based on the alert analysis. That is, the rules processing engine 134 will retrieve the rules for a particular situation to generate summary text. For example, if the alerting rules analysis from step 188 indicates that the AT/AF episode has a burden above a first threshold and below a second threshold (e.g., ≥1% and <90%), the rules processing engine 134 will retrieve a summary text for this range of the burden parameter and generate the structured report in step 190 with a summary text and numeric data (i.e., Burden ≥1% and <90%) with a yellow alert background. However, if the alerting rules analysis from step 188 indicates that the AT/AF episode has a burden above the second threshold (e.g., 90%), the rules processing engine 134 will retrieve a summary text for this range of the burden parameter and generate the structured report in step 190 with a summary text and numeric data (i.e., Burden >90%) with a red alert background. Another attribute that may be relevant is the patient's medication status. If the patient is on anti-coagulants, the alerting and reporting rules 136 may alter the decision made by the rules processing engine 134 so that a Burden % and <90% is reported with a green background as non-life threatening due to the patient medication status.

In step 192, the rules processing engine 134 sends report with the structured data and data analysis results to the reporting module 142 and the process ends at 194.

The system 100 further comprises the reporting module 142 and user interface 144. The reporting module 142 does not create the reports; those are generated by the rules processing engine 134. However, the reporting module 142 serves as a repository for reports generated by the rules processing engine 134 and the user interface 144 permits the clinic user (e.g., technician) to triage, search view, and edit the information provided by the rules processing engine 134 before sending the report to a provider for their review and signature. Other individuals, such as the provider, can also use the user interface 144 to perform similar actions on the reports in the reporting module 142.

In the embodiment described above, the rules processing engine 134 applies data from the alerting and reporting rules 136 that has been created by medical experts. In an alternative embodiment, the system 100 can apply artificial intelligence (AI) principles to machine learning. One or more sets of training data are provided to permit the rules processing engine 134 to “learn” how to determine alerting thresholds and apply those thresholds to the normalized data. The training set(s) include the various attributes described above. In an exemplary embodiment, the default alert rules described above can be created by the AI. The AI principles can be used by the rules processing engine 134 to thereby create a machine-generated report with the appropriate structured report (e.g., summary text, numeric data, and background colors) described above. In one example, the machine-generated report is a “draft” report, and the technician still provides a review of the machine-generated report prior to delivery of the report to the provider. In another exemplary embodiment, the rules processing engine 134 to thereby create a machine-generated “final” report for delivery directly to the provider.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

The invention claimed is:

1. A system for remote medical data sensing and analysis, comprising:
a data acquisition module, coupled to a plurality of input devices that each generate a set of medical data, and configured to receive the set of medical data generated by each respective one of the plurality of input devices;
a data normalization module configured to receive the sets of medical data and convert each set of medical data from the plurality of input devices to a corresponding set of standardized medical data having a uniform standard, the uniform standard comprising attribute identification data for an attribute related to each of a plurality of medical episodes and parameter identification data for one or more parameters associated with each of the attributes;
an alerting and reporting rules module configured to store a set of alerting rules that define a plurality of alerting thresholds and to store a set of structured summary texts for use in a report;
a rules processing engine configured to analyze each set of standardized medical data to identify attributes reported therein, and to analyze each of the one or more parameters associated with each of the identified attributes, the rules processing engine being further configured to apply the stored set of alerting rules to the one or more parameters associated with each of the identified attributes to apply one or more of the plurality of alerting thresholds to the one or more parameters associated with each of the identified attributes and to select at least one of the structured summary texts for use with the one or more of the plurality of alerting thresholds, to thereby generate a report of each identified attribute; and
a reporting module configured to receive and store the report of each identified attribute, the reporting module including a user interface to permit searching and editing of the report.

2. The system of claim 1 wherein the set of alerting rules and/or set of structured summary texts in the alerting and reporting rules module have initial default values and a manufacturer of one of the plurality of input devices has manufacturer-selected values for the set of alerting rules and/or structured summary texts, wherein the alerting and reporting rules module overrides the manufacturer-selected values for the set of alerting rules and/or structured summary texts and uses the initial default values for the set of alerting rules and/or structured summary texts.

3. The system of claim 1 for use in a clinic wherein the set of alerting rules and/or structured summary texts have initial default values, the system further comprising a rules configuration process to permit the clinic to enter clinic-selected values for the set of alerting rules and/or structured summary texts, the clinic-selected values overriding the initial default values for the set of alerting rules and/or structured summary texts.

4. The system of claim 1 wherein the set of alerting rules is generated using artificial intelligence.

5. The system of claim 4 wherein the set of alerting rules has initial default values, the default values being the set of alerting rules generated using artificial intelligence.

6. The system of claim 1 wherein the data normalization module is further configured to identify each of a plurality of attribute types from the set of medical data generated by each respective one of the plurality of input devices, and to assign a unique attribute identifier, as the attribute identification data, to each of the plurality of attribute types, the alerting and reporting rules module being configured to store the set of alerting rules and the set of structured summary texts in association with the unique attribute identifier.

7. The system of claim 5 wherein each of the plurality of identified attribute types has one or more associated parameters, the data normalization module being further configured to assign a unique parameter identifier, as parameter identification data, to each of the one or more associated parameters, the alerting and reporting rules module being configured to store each of the one or more associated parameters in association with the unique parameter identifier.

8. The system of claim 5 wherein the data normalization module is further configured to identify each of a plurality of attribute types from the set of medical data from each respective one of the plurality of input devices and to assign an identical attribute identifier to an identical attribute type from each set of medical data from each respective one of the plurality of input devices.

9. The system of claim 1 wherein the attributes further comprise patient information, the system further comprising a patient data storage area configured to store the patient information in association with unique patient identification data.

10. The system of claim 1 wherein the attributes further comprise device information related to one of the plurality of input devices wherein the data normalization module is configured to receive the device information attribute and convert the device information attribute to a corresponding set of data having the uniform standard.

11. The system of claim 1 wherein each of the plurality of input devices is associated with a respective manufacturer, and the set of medical data generated by each of the plurality of input devices is delivered to a manufacturer storage device of the respective manufacturer for storage therein.

12. The system of claim 10 wherein the data acquisition module is further configured to receive the set of medical data generated by each respective one of the plurality of input devices by communicating with the manufacturer storage device of the respective manufacturer to retrieve the set of medical data stored therein.

13. The system of claim 1, further comprising a plurality of communication links between each of the input devices and the data acquisition module, each of the plurality of communication links being a secure communication link.

14. A method for remote medical data sensing and analysis, comprising:
receiving a set of medical data generated by each respective one of a plurality of input devices, the sets of medical data including an attribute related to each of a plurality of medical episodes detected by one or more of the plurality of input devices wherein the sets of medical data are in a form selected by each of the plurality of input devices;
converting each set of medical data from the form selected by each respective one of the plurality of input devices to a corresponding set of standardized medical data having a uniform standard, the uniform standard comprising attribute identification data for the attribute related to each of the plurality of medical episodes and parameter identification data for one or more parameters associated with each of the attributes;
storing a set of alerting rules that define a plurality of alerting thresholds and a set of structured summary texts for use in a report;
analyzing each set of standardized medical data to identify attributes reported therein, and analyzing each of the one or more parameters associated with each of the identified attributes;
applying the stored set of alerting rules to the one or more parameters associated with each of the identified attributes and applying one or more of the plurality of alerting thresholds to the one or more parameters;
selecting at least one of the structured summary texts for use with the one or more of the plurality of alerting thresholds, to thereby generate a report of each identified attribute; and
receiving and storing the report of each identified attribute in a report storage module, the report storage module including a user interface to permit searching and editing of the report.

15. The method of claim 13 wherein the stored set of alerting rules and/or the stored set of structured summary texts have initial default values and a manufacturer of one of the plurality of input devices has manufacturer-selected values for the set of alerting rules and/or structured summary texts, the method further comprising overriding the manufacturer-selected values for the set of alerting rules and/or structured summary texts and using the initial default values for the set of alerting rules and/or structured summary texts.

16. The method of claim 13 for use in a clinic wherein the stored set of alerting rules and/or the stored set of structured summary texts have initial default values and the clinic has clinic-selected values for the set of alerting rules and/or structured summary texts, the method further comprising overriding the initial default values for the set of alerting rules and/or structured summary texts and using the clinic-selected values for the set of alerting rules and/or structured summary texts.

17. The method of claim 13 wherein the set of alerting rules is generated using artificial intelligence.

18. The method of claim 16 wherein the set of alerting rules has initial default values, the default values being the set of alerting rules generated using artificial intelligence.

19. The method of claim 13 wherein converting each set of medical data comprises identifying each of a plurality of attribute types from the set of medical data, assigning a unique attribute identifier, as the attribute identification data, to each of the plurality of attribute types, and wherein storing the set of alerting rules and the set of structured summary texts comprises storing the set of alerting rules and the set of structured summary texts in association with the unique attribute identifier to thereby identify the attribute to which individual ones of the set of alerting rules and the set of structured summary texts are applied.

20. The method of claim 18 wherein each of the plurality of identified attribute types has one or more associated parameters, the data normalization module further assigning a unique parameter identifier, as parameter identification data, to each of the one or more associated parameters, and wherein storing the set of alerting rules and the set of structured summary texts comprises storing each of the one or more associated parameters in association with the unique parameter identifier.

21. The method of claim 13 wherein the attributes further comprise patient information, the method further comprising storing the patient information in association with unique patient identification data.

22. The method of claim 13 wherein the attributes further comprise device information related to one of the plurality of input devices, the method further comprising receiving the device information attribute and converting the device information attribute to a corresponding set of data having the uniform standard.

23. The method of claim 13 wherein each of the plurality of input devices is associated with a respective manufacturer, and the set of medical data generated by each of the plurality of input devices is delivered to a manufacturer storage device of the respective manufacturer for storage therein, the method further comprising receiving the set of medical data generated by each respective one of the plurality of input devices by communicating with the manufacturer storage device of the respective manufacturer and retrieving the set of medical data stored therein.

24. A computer-readable medium for remote medical data sensing and analysis, comprising a non-transitory computer-readable medium containing a set of computer instructions that, if executed by one or more processors, causes the one or more processors to:
receive a set of medical data generated by each respective one of a plurality of input devices, the sets of medical data including an attribute related to each of a plurality of medical episodes detected by one or more of the plurality of input devices wherein the sets of medical data are in a form selected by each of the plurality of input devices;
convert each set of medical data from the form selected by each respective one of the plurality of input devices to a corresponding set of standardized medical data having a uniform standard, the uniform standard comprising attribute identification data for the attribute related to each of the plurality of medical episodes and parameter identification data for one or more parameters associated with each of the attributes;
store a set of alerting rules that define a plurality of alerting thresholds and a set of structured summary texts for use in a report;
analyze each set of standardized medical data to identify attributes reported therein, and analyze each of the one or more parameters associated with each of the identified attributes;
apply the stored set of alerting rules to the one or more parameters associated with each of the identified attributes and apply one or more of the plurality of alerting thresholds to the one or more parameters;
select at least one of the structured summary texts for use with the one or more of the plurality of alerting thresholds, to thereby generate a report of each identified attribute; and
receive and store the report of each identified attribute in a report storage module, the report storage module including a user interface to permit searching and editing of the report.
